SEQUENCE LISTING (26 sequences)

SEQ ID NO: 1 - DNA, 30 bp, synthetic construct
  source 1..30: mol_type = other DNA
tttgagcgtgatgtcttctcaccaatcttc

SEQ ID NO: 2 - DNA, 30 bp, synthetic construct
  source 1..30: mol_type = other DNA
ctacctgatttgaggcgacaacaaaacgaa

SEQ ID NO: 3 - DNA, 30 bp, synthetic construct
  source 1..30: mol_type = other DNA
cctgtttgagcgtgatgtcttctcaccaat

SEQ ID NO: 4 - DNA, 30 bp, synthetic construct
  source 1..30: mol_type = other DNA
taagttcagcgggtagtcctacctgatttg

SEQ ID NO: 5 - DNA, 30 bp, synthetic construct
  source 1..30: mol_type = other DNA
catgcctgtttgagcgtgatgtcttctcac

SEQ ID NO: 6 - DNA, 30 bp, synthetic construct
  source 1..30: mol_type = other DNA
agttcagcgggtagtcctacctgatttgag

SEQ ID NO: 7 - DNA, 30 bp, synthetic construct
  source 1..30: mol_type = other DNA
tgcctgtttgagcgtgatgtcttctcacca

SEQ ID NO: 8 - DNA, 30 bp, synthetic construct; identical to SEQ ID NO: 4
  source 1..30: mol_type = other DNA

SEQ ID NO: 9 - DNA, 30 bp, synthetic construct
  source 1..30: mol_type = other DNA
accaatcttcgcggtggcgttgcattcaca

SEQ ID NO: 10 - DNA, 30 bp, synthetic construct; identical to SEQ ID NO: 6
  source 1..30: mol_type = other DNA

SEQ ID NO: 11 - DNA, 30 bp, synthetic construct
  source 1..30: mol_type = other DNA
gatcattattgatattttgcatacacactg

SEQ ID NO: 12 - DNA, 30 bp, synthetic construct
  source 1..30: mol_type = other DNA
agatccgttgttgaaagttttctttatagt

SEQ ID NO: 13 - DNA, 30 bp, synthetic construct
  source 1..30: mol_type = other DNA
tattgatattttgcatacacactgatttgg

SEQ ID NO: 14 - DNA, 30 bp, synthetic construct
  source 1..30: mol_type = other DNA
accaagagatccgttgttgaaagttttctt

SEQ ID NO: 15 - DNA, 30 bp, synthetic construct
  source 1..30: mol_type = other DNA
gatattttgcatacacactgatttggattt

SEQ ID NO: 16 - DNA, 30 bp, synthetic construct
  source 1..30: mol_type = other DNA
caatgtgcgttcaaagattcgatgattcac

SEQ ID NO: 17 - DNA, 30 bp, synthetic construct
  source 1..30: mol_type = other DNA
ctataaagaaaactttcaacaacggatctc

SEQ ID NO: 18 - DNA, 30 bp, synthetic construct
  source 1..30: mol_type = other DNA
ttcgatgattcacgtctgcaagtcatacta

SEQ ID NO: 19 - DNA, 30 bp, synthetic construct
  source 1..30: mol_type = other DNA
actgatttggattttaaaactaacccaacg

SEQ ID NO: 20 - DNA, 30 bp, synthetic construct
  source 1..30: mol_type = other DNA
ttcaaagattcgatgattcacgtctgcaag

SEQ ID NO: 21 - DNA, 45 bp, synthetic construct
  source 1..45: mol_type = other DNA
actgatttggattttaaaactaacccaacgtaagttcaactaaac

SEQ ID NO: 22 - DNA, 30 bp, synthetic construct
  source 1..30: mol_type = other DNA
aaggatcattattgatattttgcatacaca

SEQ ID NO: 23 - DNA, 30 bp, synthetic construct
  source 1..30: mol_type = other DNA
tcattattgatattttgcatacacactgat

SEQ ID NO: 24 - DNA, 30 bp, synthetic construct
  source 1..30: mol_type = other DNA
attgatattttgcatacacactgatttgga

SEQ ID NO: 25 - DNA, 30 bp, synthetic construct
  source 1..30: mol_type = other DNA
tattttgcatacacactgatttggatttta

SEQ ID NO: 26 - DNA, 30 bp, synthetic construct; identical to SEQ ID NO: 20
  source 1..30: mol_type = other DNA

(19) United States
(12) Patent Application Publication
(10) Pub. No.: US 2025/0115968 A1
(43) Pub. Date: Apr. 10, 2025

(54) CONSTANT TEMPERATURE NUCLEIC ACID AMPLIFICATION RAA PRIMER PROBE COMBINATION FOR DETECTING CANDIDA AURIS AND USE THEREOF

(71) Applicant: ACADEMY OF MILITARY MEDICAL SCIENCES, Beijing (CN)

(72) Inventors: Ying YANG, Beijing (CN); Xinran ZHANG, Beijing (CN); Teng MA, Beijing (CN); Yuchen WANG, Beijing (CN)

(21) Appl. No.: 18/729,219

(22) PCT Filed: Oct. 8, 2023
(86) PCT No.: PCT/CN2023/123418

§ 371 Date: Jul. 16, 2024

(30) Foreign Application Priority Data
Oct. 9, 2022 (CN) 202211225468.1

(51) Int. Cl. C12Q 1/6895; C12Q 1/6844

(52) U.S. Cl. CPC C12Q 1/6895; C12Q 1/6844

(57) ABSTRACT

The present invention discloses a constant temperature nucleic acid amplification RAA primer probe combination for detecting Candida auris and use thereof are provided. The primer probe combination has high amplification efficiency, strong specificity, and no obvious false positive, and can effectively distinguish Candida auris from three closely related bacteria frequently misdiagnosed in a clinical microorganism identification system. Based on an RAA-lateral flow chromatography technology, the present invention establishes a method for rapidly detecting Candida auris can be used for detecting an actual sample after passing the specificity and sensitivity evaluation, and provides a rapid, sensitive, reliable, and effective new method for on-site rapid detection of Candida auris. The method does not need complex instruments, and is particularly suitable for rapid screening and detection of Candida auris in basic laboratories and quarantine sites.

### CROSS-REFERENCE TO RELATED APPLICATIONS

This present application is a U.S. national stage entry of PCT International Application No. PCT/CN2023/123418, filed on Oct. 8, 2023, which claims priority to the following application document: application No. 2022112254681 entitled “CONSTANT TEMPERATURE NUCLEIC ACID AMPLIFICATION RAA PRIMER PROBE COMBINATION FOR DETECTING CANDIDA AURIS AND USE THEREOF” filed on Oct. 9, 2022, the content of which is incorporated herein by reference in its entirety.

### INCORPORATION OF SEQUENCE LISTING

This application contains a sequence listing submitted in Computer Readable Form (CRF). The CRF file contains the sequence listing entitled “PA6300011-SequenceListing.xml”, which was created on Jun. 13, 2024, and is 24,061 bytes in size. The information in the sequence listing is incorporated herein by reference in its entirety.

### TECHNICAL FIELD

The present application belongs to the technical field of pathogenic microorganism detection, and particularly relates to a constant temperature nucleic acid amplification RAA primer probe combination for detecting Candida auris and use thereof.

### BACKGROUND

Candida auris is a pathogenic fungus first found in the secretions of the external auditory canal of a Japanese patient in 2009, and has been reported so far in over 40 countries of six continents other than Antarctica. Candida auris infection can occur in people of all ages. The infection occurs most frequently in the elderly, and occasionally in neonates and children. Common hosts of Candida auris can be classified into three categories: the first is the population suffering from severe immunodeficiency diseases or having a weak autoimmune ability, the second is a patient with open surgery or an invasive catheter, and the third is the population using broad-spectrum antibacterial drugs for treatment. The fungus mainly causes persistent and invasive infection and has multiple drug resistance to common antifungal drugs such as fluconazole. The mortality rate of infected patients is up to 30%-60%. The fungus can be quickly dispersed and can cause outbreak of nosocomial infection, thereby being called as “super fungus”. Candida auris, as a newly-appeared yeast with multiple drug resistance, has a relatively large genome diversity, multiple drug resistance, and high lethality rate. It is easy to spread and infect patients in hospitals, thereby causing nosocomial outbreak infection which is frequently seen in candidemia. The current common phenotype identification and microorganism identification systems can not accurately identify Candida auris, often causing the identification error of the strain in a clinical microbiology laboratory, further causing a misdiagnosis, which makes it difficult to clinically prevent and control the propagation of Candida auris timely.

Accurate identification of pathogens is a prerequisite for the treatment of any infectious disease, and only timely and accurate diagnosis can ensure the fastest reasonable treatment and effectively control the risk of further infection, thereby reducing the chance of outbreak of infectious diseases in the environment. Candida auris has a general similarity in phenotype and physiology and biochemistry to other Candida, thus making it susceptible to misdiagnosis during clinical diagnosis. Therefore, diagnosis of infections of this fungus requires use of relatively special biochemical or molecular biology detection means, for example, detection for specific proteins based on matrix-assisted laser desorption/ionization time of flight mass spectrometry (MALDI-TOF MS) or analysis identification based on sequences of internal transcribed spacer (ITS) and D1/D2 region of 28S rDNA (ribosomal DNA) in a ribosome. Among them, although MALDI-TOF MS can be used for identification of Candida auris, this method relies on database updates, not all MALDI-TOF MS devices can be used for Candida auris detection, and it must be based on pure cultures and the types of proteins available for strain-specific identification are limited. Therefore, it is often limited in practical applications. In addition, the methods for Candida auris identification and diagnosis require use of corresponding equipment, high-cost additional equipment, and technically trained professionals. The identification cost is high, and many medical institutions find it difficult to afford.

In addition, the conventional fungus identification means, including Candida chromogenic culture media and microorganism commercial detection systems, are time-consuming and labor-consuming, and have a low positive rate and high error rate. Among them, the most commonly used clinical microorganism identification systems, such as VITEK2, API-20C AUX, and BD yeast identification systems, often misdiagnose Candida auris as other closely related Candida such as C. haemulonii, C. duobushaemulonii, C. pseudohaemulonii, C. guilliermondii, and C.parapsilosis, resulting in extremely high misdiagnosis and missed diagnosis rates for patients with Candida auris infections. 3 nosocomial fungal infections caused by Candida auris were reported in 2011 in Korea, and the study showed that Candida auris was often falsely identified as Candida haemulonii and Rhodotorula glutinis by commercial identification systems such as VITEK and API-20C AUX. ITS sequencing in a comprehensive study in India confirmed that 90 of 102 strains previously identified by the VITEK system as Candida haemulonii or Candida famata should be Candida auris, with a false identification rate of up to 88.2%. It can be seen that the existing diagnostic tools are costly, have low accuracy, and may result in the incidence of Candida auris and its relatives being underestimated globally, especially in areas with restricted medical resources (e.g. Africa and Southeast Asia). This also explains to a certain extent the reasons why the existing outbreak and prevalence and individual cases of the infection are from developed countries (e.g. USA, UK, and Germany) or from medically developed cities in low income areas (e.g. New Delhi, India).

Isothermal nucleic acid amplification technology is a general term for a class of molecular biology techniques that have been newly developed in recent years. The technology can amplify a specific DNA or RNA at a specific temperature. The technology is simpler and more convenient than the PCR technology in the aspects of actual operation and instrument requirements, gets rid of the dependence on fine equipment, greatly shortens the reaction time, and shows good application prospects in clinical and on-site rapid diagnosis. Various isothermal amplification methods have been developed, such as nucleic acid sequence-dependent amplification (NASBA), strand displacement amplification (SDA), loop-mediated isothermal amplification (LAMP), helicase-dependent amplification (HAD), recombinase polymerase amplification (RPA), recombinase aided amplification (RAA), transcription-based amplification system (TAS), and rolling circle amplification (RCA). Among them, RAA is a novel nucleic acid constant temperature amplification technology, which can perform single-molecule nucleic acid detection at normal temperature within 20 minutes. The technology does not depend on expensive laboratory equipment and professional operators, which has important significance and good application prospects for monitoring and identifying Candida auris in regions with undeveloped economic and sanitary conditions and disease outbreak sites. However, no relevant report on the application of the RAA technology in the detection of Candida auris exists at present, and no appropriate and efficient RAA primer probe set can be used for the detection of Candida auris. Therefore, how to overcome the above disadvantages of the prior art and establish a Candida auris detection method with high specificity, accuracy, sensitivity, rapidness, convenience, and low cost is one of the technical problems to be solved urgently in the field.

### SUMMARY

Aiming at the above-mentioned disadvantages in the prior art, the present application is intended to provide a constant temperature nucleic acid amplification RAA primer probe combination for detecting Candida auris and use thereof.

The above purpose of the present application is implemented by the following technical schemes:

In a first aspect of the present application, provided is a constant temperature nucleic acid amplification RAA primer probe combination for detecting Candida auris.

Further, the primer probe combination comprises an upstream primer, a downstream primer, and a probe;
the upstream primer has a nucleotide sequence of F10′-1: 5′-AAGGATCATTATTGATATTTTGCATACACA-3′;
the downstream primer has a nucleotide sequence of R10′: 5′-Biotin-TTCAAAGATTCGATGATTCACGTCTGCAAG-3′;
the probe has a nucleotide sequence of P: 5′-FAM-ACTGATTTGGATTTTAAAACTAACCCAACG [THF] TAAGTTCAACTAAAC-C3spacer-3′.

In a specific embodiment of the present application, the upstream primer has a nucleotide sequence set forth in SEQ ID NO: 22, and the downstream primer has a nucleotide sequence set forth in SEQ ID NO: 26 and the 5′ end thereof is modified by biotin; the probe has a nucleotide sequence set forth in SEQ ID NO: 21, the 5′ end thereof is modified by a fluorescent group (FAM), the 3′ end thereof is blocked by a blocking group (C3-spacer), and the 31st base is replaced by tetrahydrofuran ([THF]).

In some embodiments, primers and/or probes corresponding to sequences having 70% or more homology to the sequences of the primer and/or the probe described in the present application are also included within the protection scope of the present application, i.e., it can be understood as follows: primers and/or probes obtained after modification based on the primer and/or the probe provided by the present application also fall within the protection scope of the present application.

In some embodiments, the present application has no particular limitation on the modification group (e.g., biotin) included in the primer, and the fluorescent group (e.g., FAM), the blocking group (e.g., C3-spacer), and the replacement group (e.g., tetrahydrofuran) included in the probe. Primers and/or probes obtained by replacing one or more of the above groups by other replaceable groups known to those skilled in the art are also included within the protection scope of the present application.

In some embodiments, the modification group modifying the primer includes, but is not limited to: biotin, phosphorylation, digoxin, sulfhydryl, Spacer, thio, deoxyuracil, deoxyinosine, and the like; the fluorescent group in the probe includes, but is not limited to: FAM, HEX, VIC, ROX, Cy5, TET, and the like; the blocking group in the probe includes, but is not limited to: C3-spacer, phosphate, biotin-TEG, and the like.

In the present application, the Candida auris is not particularly limited, including but not limited to: C.auris, C.haemulonii, C.pseudohaemulonii, C.duobushaemulonis, C.rugosa, C.albicans, C.neoformans, C.parapsilosis, C.glabrata, C.guilliermondii, E. coli, S.aureus, E.faecalis, C.tropicalis, C.krusei, C.dubliniensis, K.pneumoniae, and the like. Candida auris from any strain source and/or any strain type is within the protection scope of the present application.

In a second aspect of the present application, provided is a kit for detecting Candida auris.

Further, the kit comprises a primer probe mixture consisting of the primer probe combination according to the first aspect of the present application.

Further, the kit further comprises A Buffer, B Buffer, an RAA reaction dry powder reagent, and ddH₂O; preferably, the A Buffer is 20% PEG; preferably, the B Buffer is 280 mM MgAc; preferably, the RAA reaction dry powder reagent comprises the following components: dNTP, SSB protein, recA recombinase protein or Rad51, Bsu DNA polymerase, Tricine, PEG, dithiothreitol, creatine kinase, and Nfo endonuclease; more preferably, concentrations of each of the components in the RAA reaction dry powder reagent are: 1 mmol/L dNTP, 90 ng/μL SSB protein, 120 ng/μL recA recombinase protein or 30 ng/μL Rad51, 30 ng/μL Bsu DNA polymerase, 100 mmol/L Tricine, 20% PEG, 5 mmol/L dithiothreitol, 100 ng/μL creatine kinase, and Nfo endonuclease.

Further, the kit further comprises a lateral flow strip.

In some embodiments, the primer and the probe disclosed in the present application may be provided in the form of a kit for detecting Candida auris. In such a kit, a suitable amount of one or more probes and/or primers (e.g., the primer and the probe disclosed in the first aspect of the present application) are provided in one or more containers, or immobilized on a substrate. The nucleic acid probe and/or primer may be provided to suspend in an aqueous solution, or for example as a freeze-dried or lyophilized powder. The container in which the nucleic acid is provided may be any conventional container capable of containing the provided form, such as a microcentrifuge tube, ampoule, or bottle. The kit may comprise a labeled or unlabeled probe for detecting nucleotide sequences of Candida auris. In certain applications, one or more primers, e.g., primer pairs, may be provided in a pre-measured single use amount in a separate, typically disposable, tube or equivalent container. With such an arrangement, a sample for testing for the presence of Candida auris nucleic acid can be added to a separate tube and directly subjected to amplification.

The amount of nucleic acid primers provided in the kit may be any suitable amount, and may depend on the target market to which the product is directed. For example, if the kit is suitable for research or clinical use, the amount of each nucleic acid primer provided may be an amount sufficient to initiate several PCR amplification reactions. General guidance in determining suitable amounts can be found in documents of Innis et al., Sambrook et al., and Ausubel et al. The kit may comprise two or more primers to facilitate PCR amplification of a relatively large number of Candida auris nucleotide sequences.

In some embodiments, the kit may comprise a necessary reaction reagent for performing a PCR amplification reaction, including a DNA sample preparation reagent, a suitable buffer (e.g., polymerase buffer), a salt (e.g., magnesium chloride), and deoxyribonucleotides (dNTPs), and the resulting kit after a simple replacement of the type of the conventional reaction reagents is also included within the protection scope of the present application. In addition, one or more control sequences for the PCR reaction may also be provided in the kit.

In a third aspect of the present application, provided is a method for detecting Candida auris based on RAA-lateral flow chromatography technology.

Further, the method comprises the following steps:
(1) extracting DNA of a test sample;
(2) performing an RAA amplification reaction using the DNA of the test sample as a template to give an amplification product, wherein an upstream primer of the RAA amplification has a nucleotide sequence of F10′-1: 5′-AAGGATCATTATTGATATTTTGCATACACA-3′; a downstream primer has a nucleotide sequence of R10′: 5′-Biotin-TTCAAAGATTCGATGATTCACGTCTGCAAG-3′; a probe has a nucleotide sequence of P 5′-FAM-ACTGATTTGGATTTTAAAACTAACCCAACG [THF] TAAGTTCAACTAAAC-C3spacer-3′;
(3) detecting the amplification product using a lateral flow strip, wherein when two red bands appear on the strip, one in a quality control area and one in a detection area, the result is positive, indicating that the sample comprises Candida auris; when only one red band appears in the quality control area of the strip and the detection area has no band, the result is negative, indicating that the sample does not comprise Candida auris.

Further, the amplification reaction in step (2) comprises the steps of: adding the upstream primer, the downstream primer, the probe, A Buffer, ddH₂O, and the template into a detection unit tube comprising an RAA reaction dry powder reagent, adding B Buffer on the tube cover, putting on the tube cover, centrifuging at low speed, and continuing to perform the amplification reaction to give the amplification product. Further, the amounts of each of the substances used are: 2 μL of 2 μM upstream primer, 2 μL of 2 μM downstream primer, 0.6 μL of 2 μM probe, 25 μL of A Buffer, 15.9 μL of ddH₂O, 2 μL of template, and 2.5 μL of B Buffer, respectively. Further, the reaction conditions of the amplification reaction in step (2) are: reacting at 37° C. for 15 min.

In the present application, it is understood by those skilled in the art that after appropriate adjustments to the reaction conditions and/or the amounts of each substance used in the amplification reaction in step (2), it is still capable of achieving the purpose of detecting Candida auris in the test sample derived from a subject. Therefore, the reaction conditions and/or the amounts of each substance of the amplification reaction described above are not intended to limit the protection scope of the present application, and the reaction conditions and/or the amounts of each substance after being properly adjusted will also fall within the protection scope of the present application as long as the purpose of detecting Candida auris in the test sample derived from the subject is capable of being achieved.

In a specific embodiment of the present application, the amplification principle of the RAA technology is as follows: the RAA technology mainly uses a recombinase, a single strand binding protein, and a DNA polymerase to amplify a target gene in a large amount. A recombinase obtained from a bacterium or a fungus can be closely bound to a primer DNA at normal temperature to form a recombinase/primer complex, which invades a DNA double-stranded nucleic acid template. At the site of invasion, the recombinase opens the double strand and the single strand simultaneously binds to the single strand opened by the recombinase, maintaining the double-stranded template in an open-strand state. The recombinase/primer complex begins scanning the double strand. When the primer finds a perfectly matching complementary sequence on the template, the recombinase/primer complex breaks down, and the DNA polymerase binds to the 3′ end of the primer, starting to synthesize a new chain. The synthesized new chain can be used as a template. Finally the amplification product is exponentially increased to complete the amplification of the target gene.

In a specific embodiment of the present application, the downstream primer and the probe are modified at the 5′ end with biotin and carboxyfluorescein (FAM) (Tsingke Biotechnology Co., Ltd., Beijing, China), respectively. The probe labeled by fluorescence is combined with the amplification product. The endonuclease in the reaction system recognizes and cuts purine-free and pyrimidine-free [THF] sites. When the probe is cut by the endonuclease, the probe and a biotin-labeled primer are amplified together to form a fragment with a fluorescence label and biotin label at two ends, which can be interpreted by adopting a lateral flow strip. The lateral flow strip is a universal nucleic acid detection strip, which can perform detection only requiring a small amount of a product, and needs to be diluted properly when the concentration of the product is too high. When the diluted product is dripped on the sample pad, two ends of the amplification product are labeled by biotin and FAM. The FAM is combined with AuNPs. When the amplification product passes through a streptavidin detection line, the biotin is combined with the streptavidin, and the other end presents a positive signal through gold nanoparticles (AuNPs). The lateral flow strip (Hangzhou Zhongce Bio-Sci & Tech Co., Ltd., Hangzhou, China) was inserted with the sample pad facing down into the diluted reaction solution for 2 minutes, and the detection result can be visually read.

In a specific embodiment of the present application, the optimal temperature and time of the amplification reaction in the RAA method are respectively 37° C. and 15 min by further optimization of reaction conditions and screening. In addition, the method has extremely high specificity and good sensitivity and can detect Candida auris genomic DNA as low as 10¹ fg/reaction, and the detection sensitivity is not influenced by the existence of other fungal DNA.

In some embodiments, the test sample is derived from a clinical sample of a subject in need thereof, including but not limited to: cells, tissues, body fluids, such as skin; mucous membranes; blood; blood derivatives or fractions, such as serum; extracted bile; tissues that are biopsied or surgically removed, including, for example, unfixed, frozen, formalin-fixed, and/or paraffin-embedded tissues; tears; milk; dandruff; surface cleaning liquid; urine; sputum; cerebrospinal fluid; prostatic fluid; pus; bone marrow aspirate; middle ear effluent, bronchoalveolar lavage, tracheal aspirate, sputum, nasopharyngeal aspirate, oropharyngeal aspirate, or saliva.

In some embodiments, the subject includes a human and a non-human animal. Non-human animals include all vertebrates (e.g., mammals and non-mammals) such as non-human primates (e.g., cynomolgus monkeys), sheep, dogs, cows, chickens, amphibians, and reptiles. In certain specific embodiments, the subject is preferably a human.

In a fourth aspect of the present application, provided is use of the primer probe combination according to the first aspect of the present application and the kit according to the second aspect of the present application in preparing a product for detecting Candida auris.

In a fifth aspect of the present application, provided is use of the primer probe combination according to the first aspect of the present application and the kit according to the second aspect of the present application in preparing a product for identifying and diagnosing Candida auris, C. persedohaemulonii, C. duobushaemulonis, and C. haemulonii.

In a sixth aspect of the present application, provided is use of the primer probe combination according to the first aspect of the present application and the kit according to the second aspect of the present application in detecting Candida auris.

In a seventh aspect of the present application, provided is use of the primer probe combination according to the first aspect of the present application and the kit according to the second aspect of the present application in identifying and diagnosing Candida auris, C. persedohaemulonii, C. duobushaemulonis, and C. haemulonii.

In specific embodiments of the present application, upon verification, it has been found in the present application that the primer probe combination provided by the present application can accurately and effectively identify and distinguish Candida auris from three types of closely related bacteria, namely C. persedohaemulonii, C. duobushaemulonis, and C. haemulonii, which are frequently misdiagnosed in a clinical microorganism identification system.

In an eighth aspect of the present application, provided is a method for diagnosing whether a subject has a Candida auris infection disease, wherein the method comprises the step of: detecting a test sample derived from the subject using the method according to the third aspect of the present application.

Compared with the prior art, the present application has the advantages and beneficial effects that:

(1) The present application provides a constant temperature nucleic acid amplification RAA primer probe combination for rapidly detecting Candida auris, wherein the primer probe combination comprises a primer pair (set forth in SEQ ID NO: 22 and SEQ ID NO: 26) and a probe (set forth in SEQ ID NO: 21). The primer pair can specifically amplify Candida auris, and does not generate cross-reaction with other pathogenic bacteria except Candida auris, namely the primer pair provided by the present application has a relatively strong specific amplification characteristics, and can significantly distinguish Candida auris from closely related bacteria, namely C.haemulonii, C.pseudohaemulonii, and C.duobushaemulonis, which are frequently misdiagnosed in a clinical microorganism identification system. In addition, the present application proves that the RAA primer probe combination has the highest amplification efficiency, the strongest specificity, and no obvious false positive through a comparison experiment, and is significantly superior to other three RAA primer probe combinations which are designed aiming at the ITS sequence of the Candida auris rDNA gene.

This technical effect is unexpected by those skilled in the art.

(2) The present application also provides a POCT (point of care testing) method for rapidly detecting Candida auris, namely a detection method (RAA-LFS) combining lateral flow strip detection (LFS) and recombinase aided amplification (RAA) technology. The DNA is extracted and purified by a simple Chelex-100 boiling method, so that the detection time of the detection system is saved. The detection does not need fluorescence detection equipment and an electrophoresis device, only needs a constant-temperature water bath pot to provide the temperature required by the reaction, and can complete effective amplification even by using body temperature to supply heat. The detection result can be obtained within 15 minutes, and the method has high specificity, high sensitivity, low instrument dependence, no need of laboratory personnel with professional training, and strong operability. The method can be used for meeting the needs of bedside diagnosis on site or the needs of remote hospitals with weak conditions, and has important significance for the rapid detection of Candida auris.

(3) The present application establishes a method for rapidly detecting Candida auris for the first time by adopting an RAA-lateral flow chromatography technology, can be used for detecting an actual blood sample after passing the specificity and sensitivity evaluation, and provides a sensitive, reliable, and effective new method for on-site rapid detection of Candida auris. The primer pair selected by the present application is obtained by screening in a large number of experiments, has good specificity, and has no cross-reaction with other pathogenic bacteria. The primer probe combination used by the present application has high amplification efficiency and strong band specificity, and can form a primer-probe heterodimer with a relatively high concentration in the detection area, so that the strip shows strong positive reaction, and the detection sensitivity is increased. The method for detecting Candida auris by combining the RAA technology and the lateral flow chromatography technology of the present application has the advantages of high sensitivity and high throughput of molecular biology detection, and good specificity and convenient operation of immunological detection, does not need complex instruments, has high detection speed, and is particularly suitable for rapid screening and detection of Candida auris in basic laboratories and quarantine sites.

### BRIEF DESCRIPTION OF THE DRAWINGS

FIG. 1 is a schematic diagram of RAA-LFS, wherein panel A is RAA-nfo probe design, panel B is binding of fluorescently labeled probe to template, and panel C is a working schematic diagram of a lateral flow strip (LFS).

FIG. 2 is a schematic diagram of the interpretation method for the detection result.

FIG. 3 is a graph showing the results of agarose gel electrophoresis of primer pairs screened by amplification performance of RAA, wherein the NTC lane is non-template control of the corresponding primer pair, the band size of DNA ladder is shown on the left side, and primer dimers are indicated by white arrows.

FIG. 4 is a graph of the results of a lateral flow strip (LFS) of recombinase aided amplification (RAA) using different primer-probe sets, wherein NTC is non-template control of the corresponding primer-probe set, the positions of the test and control lines are indicated on the right side, the template is Candida auris genomic DNA, and the reaction conditions are at 39° C. for 15 minutes.

FIG. 5 is a graph of the results of phylogenetic trees of the whole genome of Candida auris and other common pathogen Candida.

FIG. 6 is a graph of primer-probe combination targeting fragments comparing the ITS sequence fragments of three closely related strains C. persedohaemulonii, C. duobushaemulonis, and C. haemulonii with the ITS sequence sections of Candida auris and showing the strain name at the beginning of each sequence, the sequence corresponding to the primer and probe is written below the position in the alignment, the arrow indicates the direction of primer and probe extension, and the tetrahydrofuran (THF) site is indicated by “□”.

FIG. 7 is a graph showing the results of RAA-LFS detection under different reaction temperature and reaction time, wherein the temperature of the RAA reaction is shown at the top of each band, the RAA reaction time is shown on the left side of each row, the amplification template is Candida auris genomic DNA, and the positions of the control line and the test line are shown on the right side of the image.

FIG. 8 is a graph showing the results of detection of different reference strains, wherein the strain names are shown at the top of each band, NTC is non-template control, the positions of the control line and the test line are shown on the right side of the image, and the reaction is performed at 37° C. for 15 minutes.

FIG. 9 is a graph showing the results of detection of other closely related fungi and pathogenic bacteria, wherein panel A is a graph of the results of RAA-LFS detection of C.haemulonii, C. Perudohaemulonii, and C. Duobushaemulonis strains, and panel B is a graph of the results of RAA-LFS detection of other related Candida and common pathogens. The strain type is shown on the top of each band, NTC is non-template control, the positions of the control line and the test line are shown on the right side of the image, and the reaction is performed at 37° C. for 15 minutes.

FIG. 10 is a graph showing the results of sensitivity of RAA-LFS technology for detecting Candida auris, wherein panel A is a graph of LFS results of RAA amplification and different amounts of Candida auris cultures, panel B is a graph of LFS results of RAA amplification with different amounts of Candida auris genomic DNA.

FIG. 11 is a graph showing the results of sensitivity of RAA-LFS technology for detecting Candida auris under other fungal interferences, wherein panel A is a graph of LFS results of RAA amplification after addition of 10⁵ CFU/μL Candida albicans culture medium to different Candida auris cultures, and panel B is a graph of LFS results of RAA amplification with 1 ng Candida albicans genomic DNA added to the reaction in addition to Candida auris genomic DNA.

FIG. 12 is a graph of the results of the detection performance test of the RAA-LFS technology on human blood samples, wherein panel A is a graph of RAA-LFS detection results by mixing Candida auris bacterial solution with different concentrations into the human blood sample, panel B is a graph of RAA-LFS detection results after mixing Candida auris genomic DNA and human blood-extracted DNA according to the ratio of 1:10, wherein NTC is non-template control, the positions of the control line and the test line are shown on the right side of the image, and the reaction is performed at 37° C. for 15 minutes.

### DETAILED DESCRIPTION

Unless otherwise indicated, technical terms referred to herein are generally used according to their conventional usage. The definition of commonly used terms in molecular biology can be found in Benjamin Lewin, Genes VII (Oxford University Press, 1999), The Encyclopedia of Molecular Biology (BlackwellScience Ltd., 1994) edited by Kendrew et al., Molecular Biology and Biotechnology: A Comprehensive Desk Reference (VCH Publishers, Inc., 1995) edited by Robert A. Meyers et al., and other similar references.

The term “comprise” or “include” as used herein, is intended to mean that the specified elements are included, but not excluding others. For example, “the kit comprises a primer probe mixture consisting of the primer probe combination” means “comprises a primer probe mixture consisting of the primer probe combination” without excluding other elements.

The term “amplification” as used herein refers to the process for increasing the copy number of a nucleic acid molecule. The resulting amplification product is referred to as an “amplicon”. Amplification of a nucleic acid molecule (e.g., a DNA or RNA molecule) refers to the use of techniques to increase the copy number of the nucleic acid molecule in a sample. An example of amplification is the polymerase chain reaction (PCR), wherein a sample is contacted with a pair of oligonucleotide primers under conditions that allow for hybridization of the primers to nucleic acid templates in the sample. The primers are extended under suitable conditions, dissociated from the template, re-annealed, extended, and dissociated to amplify the copy number of the nucleic acid. This cycle can be repeated. The amplification products can be characterized by techniques such as electrophoresis, restriction endonuclease cleavage patterns, oligonucleotide hybridization or ligation, and/or nucleic acid sequencing.

Examples of other in vitro amplification techniques include quantitative real-time PCR, reverse transcriptase PCR (RT-PCR), real-time PCR, real-time reverse transcriptase PCR (rt RT-PCR), nested PCR, strand-displacing amplification (see U.S. Pat. No. 5,744,311), nontranscribed isothermal amplification (see U.S. Pat. No. 6,033,881), repair strand reaction amplification (see WO 90/01069), ligase chain reaction amplification (see European Patent Publication EP-A-320 308), gap-fill ligase chain reaction amplification (see U.S. Pat. No. 5,427,930), coupled ligase detection and PCR (see U.S. Pat. No. 6,027,889), NASBA™ RNA nontranscribed amplification (see U.S. Pat. No. 6,025,134), or the like.

The term “primer” as used herein refers to a short nucleic acid molecule, such as a DNA oligonucleotide, e.g., a sequence of at least 15 nucleotides, that can anneal to a complementary target nucleic acid molecule by nucleic acid hybridization to form a hybrid between the primer and the target nucleic acid strand. The primer can be extended along the target nucleic acid molecule by a polymerase. Thus, primers can be used to amplify a target nucleic acid molecule (e.g., a Candida auris nucleic acid sequence), wherein the sequence of the primer is specific for the target nucleic acid molecule, and thus, for example, the primer will hybridize to the target nucleic acid molecule under hybridization conditions with very high stringency. The specificity of a primer increases with its length. Thus, for example, a primer comprising 30 consecutive nucleotides will anneal to a target sequence with greater specificity than a corresponding primer of only 15 nucleotides. Thus, to obtain higher specificity, probes and primers that comprise at least 15, 20, 25, 30, 35, 40, 45, 50 or more consecutive nucleotides can be selected.

Methods for preparing and using primers are described in, for example, Sambrook et al., Molecular Cloning: A Laboratory Manual (Cold SpringHarbor, New York, 1989) and Ausubel et al., Current Protocols in Molecular Biology (Greene Publ. Assoc. & Wiley-Intersciences, 1987).

The term “probe” as used herein refers to a probe comprising an isolated nucleic acid capable of hybridizing to a target nucleic acid (e.g., a Candida auris nucleic acid molecule). The probe may have a detectable label or reporter connected thereto. Typical labels include radioisotopes, enzyme substrates, cofactors, ligands, chemiluminescent or fluorescent reagents, haptens, and enzymes.

Methods for labeling and guidelines for selecting labels suitable for a variety of purposes are discussed in Sambrook et al., Molecular Cloning: A Laboratory Manual (Cold Spring Harbor Laboratory Press, 1989), and Ausubel et al., Current Protocols in Molecular Biology (Greene Publishing Associates and Wiley-Intersciences, 1987).

Probes are typically at least 20 nucleotides in length, e.g., at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, or more consecutive nucleotides that are complementary to a target nucleic acid molecule, for example, 20-60 nucleotides, 30-60 nucleotides, 20-50 nucleotides, 30-50 nucleotides, 20-40 nucleotides, or 20-30 nucleotides.

The term “homology” as used herein refers to the identity/similarity between two or more nucleic acid sequences, or two or more amino acid sequences, expressed in terms of identity or similarity between the sequences. Sequence identity can be measured in terms of percent identity; the higher the percentage, the more consistent the sequence. Homologs or orthologs of nucleic acid or amino acid sequences have a relatively high level of sequence identity/similarity when aligned using a standard method.

Methods for aligning sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al., Computer Appls. in the Biosciences 8, 155-65, 1992; Pearson et al., Meth. Mol. Bio. 24:307-31, 1994. In Altschul et al., J. Mol. Biol. 215:403-10, 1990, detailed considerations for sequence alignment methods and homology calculations are presented.

The present application will be further illustrated with reference to the following specific examples, which are illustrative only and are not to be construed as limiting the present application. It can be understood by those of ordinary skill in the art that various changes, modifications, replacements and variations can be made to these examples without departing from the principle and purpose of the present application, and the scope of the present application is defined by the claims and equivalents thereof. Unless otherwise stated, the experimental methods used in the following examples are conventional methods. Unless otherwise stated, the reagents, biomaterials and the like used in the following examples are commercially available.

### Example 1. Design and Screening of Primer Probe Set for RAA-LFS System

### 1.1. Material and Method

### 1.1.1. Strain Source

The sources of the experimental strains involved in the examples of the present application are shown in Table 1.

TABLE 1
Material

| Name of strain | No. | Source |
|---|---|---|
| C. auris | CBS10913 | Purchased from Netherlands |
| | | Microbiological Culture |
| | | Collection Center |
| C. auris | CBS12766 | Purchased from Netherlands |
| | | Microbiological Culture |
| | | Collection Center |
| C. auris | CBS12767 | Purchased from Netherlands |
| | | Microbiological Culture |
| | | Collection Center |
| C. auris | CBS12768 | Purchased from Netherlands |
| | | Microbiological Culture |
| | | Collection Center |
| C. auris | CBS12769 | Purchased from Netherlands |
| | | Microbiological Culture |
| | | Collection Center |
| C. auris | CBS12770 | Purchased from Netherlands |
| | | Microbiological Culture |
| | | Collection Center |
| C. auris | CBS12771 | Purchased from Netherlands |
| | | Microbiological Culture |
| | | Collection Center |
| C. auris | CBS12772 | Purchased from Netherlands |
| | | Microbiological Culture |
| | | Collection Center |
| C. auris | CBS12773 | Purchased from Netherlands |
| | | Microbiological Culture |
| | | Collection Center |
| C. auris | CBS12774 | Purchased from Netherlands |
| | | Microbiological Culture |
| | | Collection Center |
| C. auris | DSM105988 | Purchased from Germany |
| | | Microbiological Culture |
| | | Collection Center |
| C. auris | DSM105990 | Purchased from Germany |
| | | Microbiological Culture |
| | | Collection Center |
| C. haemulonii | CBS12436 | Purchased from Netherlands |
| | | Microbiological Culture |
| | | Collection Center |
| C. haemulonii | CBS12437 | Purchased from Netherlands |
| | | Microbiological Culture |
| | | Collection Center |
| C. haemulonii | CBS12438 | Purchased from Netherlands |
| | | Microbiological Culture |
| | | Collection Center |
| C. haemulonii | CBS12439 | Purchased from Netherlands |
| | | Microbiological Culture |
| | | Collection Center |
| C. pseudohaemulonii | CBS10004 | Purchased from Netherlands |
| | | Microbiological Culture |
| | | Collection Center |
| C. pseudohaemulonii | CBS12370 | Purchased from Netherlands |
| | | Microbiological Culture |
| | | Collection Center |
| C. pseudohaemulonii | CBS12371 | Purchased from Netherlands |
| | | Microbiological Culture |
| | | Collection Center |
| C. duobushaemulonis | CBS6915 | Purchased from Netherlands |
| | | Microbiological Culture |
| | | Collection Center |
| C. duobushaemulonis | CBS7089 | Purchased from Netherlands |
| | | Microbiological Culture |
| | | Collection Center |
| C. duobushaemulonis | CBS7798 | Purchased from Netherlands |
| | | Microbiological Culture |
| | | Collection Center |
| C. duobushaemulonis | CBS7799 | Purchased from Netherlands |
| | | Microbiological Culture |
| | | Collection Center |
| C. rugosa | ATCC10571 | Purchased from American |
| | | Culture Collection Center |
| C. albicans | ATCC10231 | Purchased from American |
| | | Culture Collection Center |
| C. neoformans | ATCC32609 | Purchased from American |
| | | Culture Collection Center |
| C. parapsilosis | ATCC22019 | Purchased from American |
| | | Culture Collection Center |
| C. glabrata | ATCC2001 | Purchased from American |
| | | Culture Collection Center |
| C. guilliermondii | ATCC6260 | Purchased from American |
| | | Culture Collection Center |
| E. coli | ATCC47076 | Purchased from American |
| | | Culture Collection Center |
| S. aureus | ATCC51811 | Purchased from American |
| | | Culture Collection Center |
| E. faecalis | ATCC29212 | Purchased from American |
| | | Culture Collection Center |
| C. tropicalis | CICC1253 | Purchased from China Center of |
| | | Industrial Culture Collection |
| C. krusei | CICC1674 | Purchased from China Center of |
| | | Industrial Culture Collection |
| C. dubliniensis | CMCC50042 | Purchased from National Center |
| | | for Medical Culture Collections |
| K. pneumoniae | BNCC330357 | Purchased from Beijing Beina |
| | | Chuanglian Biotechnology |
| | | Research Institute |

### 1.1.2. Major Instrument

The major instruments involved in the examples of the present application are shown in Table 2.

TABLE 2
Major instrument

| Name of instrument | Manufacturer | Cat No. |
|---|---|---|
| Intelligent bacteria | Hangzhou Qiwei Instrument | WGZ-XT |
| turbidity meter | | |
| Biosafety cabinet | Beijing Yataikelong Instrument | BSC-1000IIA2 |
| Benchtop centrifuge | Thermo | FRESCO21 |
| Vortex mixer | Haimen Qilinjuyu Instrument | XW-80A |
| Biochemical incubator | Beijing Zhongyiguoke | 3111 |
| Analytical balance | Shanghai Precision Instrument | FA10048 |
| Dry thermostat | Beijing Zhongyiguoke | DTH-100 |
| Mini handheld centrifuge | Beijing Donglinchangsheng | DL300 |
| | Biology | |
| Nanodrop ultramicro | Germany high-performance | MPIEN Nanophtometer |
| spectrophotometer | ultramicro spectrophotometer | NP80 |
| Constant-temperature | Wuxi Jianyi Instrument | Water both potDK-S24 |
| water bath pot HSYZ-SP | | |
| Refrigerator-freezer | Haier | |
| Infrared inoculating loop | Hangzhou Ruicheng Instrument | IS800-B |
| sterilizer | | |
| Gradient PCR Instrument | ABI | Veriti 96-Well Thermal |
| | | Cycler |
| Benchtop full-temperature | Shanghai Zhichu | ZQTY-70E |
| shaking incubator | | |
| Gel imaging system | Shanghai Qinxiang | GenoSens 2000 |
| Full-wavelength | BioTek | Epoch |
| microplate reader | | |
| Blue light detector | Beijing Liuyi Biology | Q32866 |
| Qubit 2.0 Fluorometer | invitrogen | |
| Power supply of | Beijing Liuyi Biology | DYY-6C |
| electrophoresis apparatus | | |
| Electrophoresis tank | Beijing Liuyi Biology | DYCP-31BN |
| High throughout tissue | Xianfengkeyi | CGRINDER-24 |
| grinder | | |
| Multi-specification pipette | Thermo | |
| Fluorescent quantitative | BIO-RED | CFX96 |
| PCR instrument | | |
| Upright optical | Leica | Leica DM750 |
| microscope | | |
| Inverted fluorescence | Leica | Leica DM IL LED |
| microscope | | |
| Microwave oven | Galanz | G70D20CN1P-D2 |

### 1.1.3. Major Reagent and Consumable

The major reagents and consumables involved in the examples of the present application are shown in Table 3.

TABLE 3
Major reagent and consumable

| Name of reagent | Manufacturer | Cat No. |
|---|---|---|
| Agarose | Shanghai Beijing | BY-R0100 |
| Nucleic acid dye Gold View II | Beijing Solarbio | G8142 |
| 50bp DNA Ladder | Beijing Solarbio | M1800 |
| 6× DNA loading buffer | Beijing Solarbio | D1010 |
| 50× TAE buffer | Magen | JET100300 |
| PBS pH 7.4 buffer 1× | gibco | C10010500BT |
| Dimethyl sulfoxide (DMSO) | Beijing Solarbio | D8370 |
| Triton ™ X-100 | Sigma | T8787-50ML |
| Dneasy nucleic acid extraction kit | QIAGEN GmbH | 69106 |
| TE buffer pH 8.0 | Beijing Solarbio | T1120 |
| DNA extraction reagent phenol | Beijing Solarbio | P1012 |
| chloroform = 25:24:1 (PH >7.8) | | |
| Chelate resin Chelex-100 | Beijing Fande | SS6072 |
| | Biology | |
| RAA nucleic acid amplification | Hangzhou Zhongce | S001ZC |
| reagent (basic type) | Biology | |
| RAA-nfo nucleic acid | Hangzhou Zhongce | S005ZC |
| amplification reagent (strip type) | Biology | |
| Disposable nucleic acid detection | Hangzhou Zhongce | R103ZC |
| strip | Biology | |
| 200 μL PCR tube, different EP | Axygene | |
| tubes, and pipette tip | | |
| Sabouraud's glucose solid culture | Beijing Sanyao | 18010 |
| medium | | |

### 1.1.4. Bioinformatics Software

Primer and probe design: Primer 5.0

Sequence alignment and splicing processing software: MEGA7.0 and BoEidt software Nucleotide and amino acid comparison tool: online tool of American Center for Biotechnology Information

### 1.2. Method

### 1.2.1. Strain Thawing

A glycerol-preserved strain was taken, fungi were coated on a SDA solid culture medium according to a sterile operation principle, bacteria were coated on an LB solid culture medium, and they were cultured in an inverted state in an incubator. A single clone was selected in a biosafety cabinet and cultured in a liquid culture medium.

### 1.2.2. Strain DNA Extraction

Genomic DNA from all strains was extracted using a Chelex-100 boiling method. All the extracted DNA was quantified using a Qubit 2 fluorometer (Thermo Fisher Scientific) and stored in a refrigerator at −20° C. until use. The specific steps for extracting the genomic DNA were as follows:
(1) the cultured bacterial solution was centrifuged, the supernatant was discarded, and the bacteria were collected;
(2) the Chelex-100 lysis buffer was added into the bacteria precipitate, and the mixture was boiled in a metal bath or a constant-temperature water bath pot;
(3) The crude extract after heating and boiling described above was fully centrifuged, the supernatant was taken and added with a DNA extraction phenol reagent (Beijing Solarbio Science & Technology Co., Ltd., Beijing, China) with the same volume, and the mixture was mixed well. The supernatant was pipetted after full centrifugation, and cryopreserved at −20° C. for later use after quantification with 2.0 Qubit and labeling.

### 1.2.3. RAA Detection Method Establishment

### 1.2.3.1. Primer Design

The ITS sequence of the Candida auris rDNA gene (NCBI Reference Sequence: NR_154998.1) was obtained by searching from the nucleic acid database GenBank (http://www.ncbi.nlm.nih.gov) of NCBI. The Candida auris rDNA gene ITS1 and ITS2 were specific conserved sequences of Candida auris and were taken as a target sequence. Primers and probes were designed using Primer Premier 5.0 software (Premier Biosoft International, CA, USA) according to the primer design principle, and 10 candidate primer pairs (ITS-1-ITS-10) were designed in total, as shown in Table 4. The designed primer pairs were screened using the BLAST tool (http://blast.ncbi.nlm.nih.gov/Blast.cgi) in NCBI.

The primer pair and the probe were synthesized by Tsingke.

TABLE 4
10 designed candidate primer pairs

| Name | Sequence (5′-3′) | |
|---|---|---|
| ITS-1 | F1 | TTTGAGCGTGATGTCTTCTCACCAATCTTC |
| | | (SEQ ID NO: 1) |
| | R1 | CTACCTGATTTGAGGCGACAACAAAACGAA |
| | | (SEQ ID NO: 2) |
| ITS-2 | F2 | CCTGTTTGAGCGTGATGTCTTCTCACCAAT |
| | | (SEQ ID NO: 3) |
| | R2 | TAAGTTCAGCGGGTAGTCCTACCTGATTTG |
| | | (SEQ ID NO: 4) |
| ITS-3 | F3 | CATGCCTGTTTGAGCGTGATGTCTTCTCAC |
| | | (SEQ ID NO: 5) |
| | R3 | AGTTCAGCGGGTAGTCCTACCTGATTTGAG |
| | | (SEQ ID NO: 6) |
| ITS-4 | F4 | TGCCTGTTTGAGCGTGATGTCTTCTCACCA |
| | | (SEQ ID NO: 7) |
| | R4 | TAAGTTCAGCGGGTAGTCCTACCTGATTTG |
| | | (SEQ ID NO: 8) |
| ITS-5 | F5 | ACCAATCTTCGCGGTGGCGTTGCATTCACA |
| | | (SEQ ID NO: 9) |
| | R5 | AGTTCAGCGGGTAGTCCTACCTGATTTGAG |
| | | (SEQ ID NO: 10) |
| ITS-6 | F6 | GATCATTATTGATATTTTGCATACACACTG |
| | | (SEQ ID NO: 11) |
| | R6 | AGATCCGTTGTTGAAAGTTTTCTTTATAGT |
| | | (SEQ ID NO: 12) |
| ITS-7 | F7 | TATTGATATTTTGCATACACACTGATTTGG |
| | | (SEQ ID NO: 13) |
| | R7 | ACCAAGAGATCCGTTGTTGAAAGTTTTCTT |
| | | (SEQ ID NO: 14) |
| ITS-8 | F8 | GATATTTTGCATACACACTGATTTGGATTT |
| | | (SEQ ID NO: 15) |
| | R8 | CAATGTGCGTTCAAAGATTCGATGATTCAC |
| | | (SEQ ID NO: 16) |
| ITS-9 | F9 | CTATAAAGAAAACTTTCAACAACGGATCTC |
| | | (SEQ ID NO: 17) |
| | R9 | TTCGATGATTCACGTCTGCAAGTCATACTA |
| | | (SEQ ID NO: 18) |
| ITS-10 | F10 | ACTGATTTGGATTTTAAAACTAACCCAACG |
| | | (SEQ ID NO: 19) |
| | R10 | TTCAAAGATTCGATGATTCACGTCTGCAAG |
| | | (SEQ ID NO: 20) |

### 1.2.3.2. Primer Screening System and Reaction Steps

10 pairs of the designed candidate primers were first preliminarily verified in NCBI. Primer screening was performed using the basic RAA nucleic acid amplification reagent (Hangzhou Zhongce Bio-Sci & Tech Co.,Ltd., Hangzhou, China) according to the manufacturer's instructions. The primers were preliminarily screened by amplifying target gene fragments with a non-template control. The amplification product was electrophoresed on an agarose gel to compare the amplification performance formed by the target and primer dimer in the non-template control. A primer pair showing the best amplification performance without signs of cross-dimer formation was selected.

The RAA primer screening system was 50 μL in total: into a detection unit tube comprising a reaction 10 dry powder were added 2 μL of each of 10 μM upstream and downstream primers, 25 μL of A Buffer, 13.5 μL of double distilled water, and 5 μL of Candida auris genomic DNA. After 47.5 μL in total of the system was prepared, the system was mixed well and centrifuged, and 2.5 μL of B Buffer was added on the tube cover. The tube cover was covered. The tube was turned upside down, gently shaken, and mixed well for 5-6 times. The mixture was added into the system by centrifugation for catalyzing the reaction. After centrifugation at low speed for 10 s, the mixture was incubated in a PCR instrument (or a constant-temperature metal bath or water bath pot) at 39° C. for 30 min. After the reaction was completed, 50 μL of the DNA extraction phenol reagent (Beijing Solarbio Science & Technology Co., Ltd., Beijing, China) was added to the detection unit tube. The mixture was mixed well and centrifuged at 12000 rpm/min for 5 min. 6 μL of the supernatant was taken and mixed with 1 μL of a 6× loading buffer, and then subjected to 2.5% agarose gel electrophoresis. The results were observed by a gel imaging system after the electrophoresis was completed.

### 1.2.3.3. Primer and Probe Design Suitable for RAA-Nfo

Primers and probes were designed according to the manufacturer's instructions in the RAA-nfo nucleic acid amplification reagent (strip type) kit (Hangzhou Zhongce Bio-Sci & Tech Co.,Ltd., Hangzhou, China) using Primer Premier 5.0 software (Premier Biosoft International, CA, USA).

### 1.2.3.4. Screening System of Primer-Probe Set and RAA Reaction Steps

Based on the above primer screening results, a candidate probe for RAA-nfo detection was obtained by extending a forward primer F10 by 16 bp at the 3′ end. All possible cross dimers generated by the probe and the reverse primer were predicted. Finally, four new upstream primers for RAA-nfo detection were designed in the upstream sequence of the probe, and the 5′ end of the original downstream primer sequence was modified with biotin to be used as the downstream primer for RAA-nfo detection. The primer probe sequences designed as described above are shown in Table 5. In the table, the FAM is a fluorescent group, the THF is tetrahydrofuran, and the C3-spacer is a blocking group.

TABLE 5
4 designed candidate primer-probe combinations

| Name | | Sequence (5′-3′) |
|---|---|---|
| ITS- | P | FAM- |
| 10′ | | ACTGATTTGGATTTTAAAACTAACCCAACG[THF] |
| | | TAAGTTCAACTAAAC C3spacer |
| | | (SEQ ID NO: 21) |
| | F10′-1 | AAGGATCATTATTGATATTTTGCATACACA |
| | | (SEQ ID NO: 22) |
| | F10′-2 | TCATTATTGATATTTTGCATACACACTGAT |
| | | (SEQ ID NO: 23) |
| | F10′-3 | ATTGATATTTTGCATACACACTGATTTGGA |
| | | (SEQ ID NO: 24) |
| | F10′-4 | TATTTTGCATACACACTGATTTGGATTTTA |
| | | (SEQ ID NO: 25) |
| | R10′ | Biotin-TTCAAAGATTCGATGATTCACGTCTGCAAG |
| | | (SEQ ID NO: 26) |

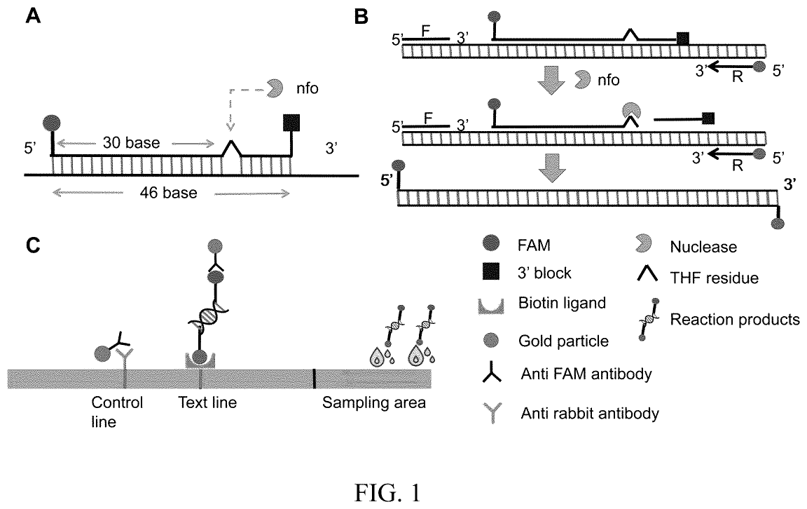

FIG. 1 is a schematic diagram of RAA-LFS. In this example, to screen for the optimal primer-probe set, screening was performed using the RAA-nfo nucleic acid amplification reagent (strip type) kit (Hangzhou Zhongce Bio-Sci & Tech Co.,Ltd., Hangzhou, China) according to the manufacturer's instructions. The RAA reaction process was as follows: The reaction system was 50 μL in total, into a detection unit tube comprising an RAA reaction dry powder reagent were added 2 μL of each of 2 μM upstream and downstream primers, 0.6 μL of a 2 μM probe, 25 μL of A Buffer (20% PEG), 15.9 μL of double distilled water (ddH₂O), and 2 μL of Candida auris genomic DNA, and 2.5 μL of B Buffer (280 mM MgAc) was added on the tube cover. The tube cover was covered. The tube was turned upside down, gently shaken, and mixed well for 5-6 times. The mixture was added into the system by centrifugation for catalyzing the reaction. After centrifugation at low speed for 10 s, the mixture was incubated in a PCR instrument (or a constant-temperature metal bath or water bath pot) at 39° C. for 15 min. After the reaction was completed, 50 μL of the amplification product was added to 300 μL of sterile water or 1×PBS solution for dilution. A disposable nucleic acid detection strip (lateral flow strip) (Hangzhou Zhongce Bio-Sci & Tech Co.,Ltd., Hangzhou, China) was inserted with the sample pad facing down into the diluted reaction solution for 2 minutes. After the sample was absorbed to the absorbent pad, the result was visually interpreted.

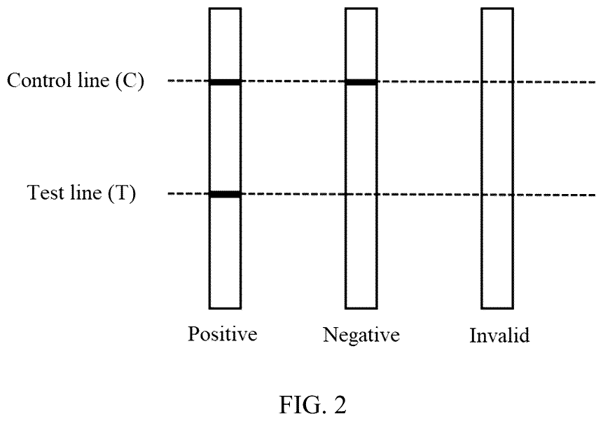

The result interpretation method is as follows: Two red bands appear on the strip, one in the quality control area (line C) and one in the detection area (line T). The positive result is that the line C and the line T appear at the same time, which indicates that the sample comprises Candida auris; the negative result is that only line C appears, and line T does not appear, which indicates that the sample does not comprise Candida auris or the quantity of Candida auris is lower than the lowest detection limit; when neither the line C nor the line T appears, the result is invalid. As shown in FIG. 2.

### 1.3. Results

1.3.1. Candida auris Primer Probe Design Results

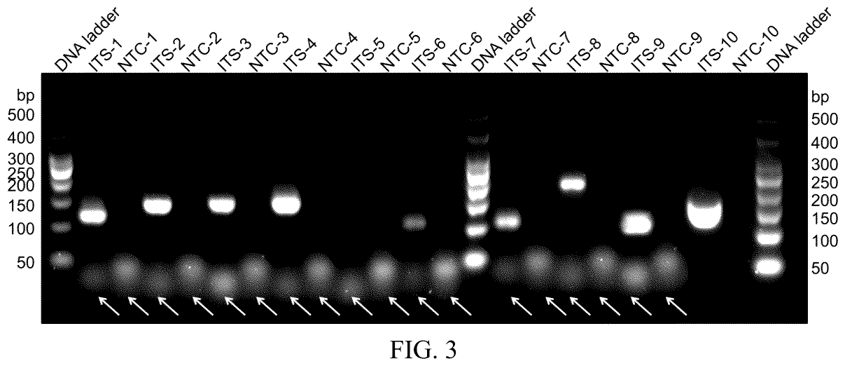

The 10 designed candidate primer pairs are shown in Table 4. The above candidate primers were preliminarily screened by amplifying target gene fragments with a non-template control. The amplification product was electrophoresed on an agarose gel to compare the amplification performance formed by the target and primer dimer in the non-template control. The results are shown in FIG. 3. According to the electrophoresis results, the primer pair ITS-10 had the brightest band and the highest amplification efficiency, and had no obvious primer dimer, so that a probe can be designed according to the positions of F10 and R10. According to the probe design principle described above, a candidate probe was obtained by extending a forward primer F10 by 16 bp at the 3′ end. All possible cross dimers generated by the probe and the reverse primer were predicted. 4 new upstream primers were designed upstream of the probe and tested and screened. The 5′ end of the probe was modified by FAM, the 3′ end thereof was blocked by C3-spacer, and the 31st base was replaced by [THF]. Four new upstream primers for RAA-nfo detection were designed in the upstream sequence of the probe, and the 5′ end of the original downstream primer sequence was modified with biotin to be used as the downstream primer for RAA-nfo detection. The results of primer and probe design suitable for RAA-nfo are shown in Table 5.

### 1.3.2. Optimal Primer-Probe Set Screening Results

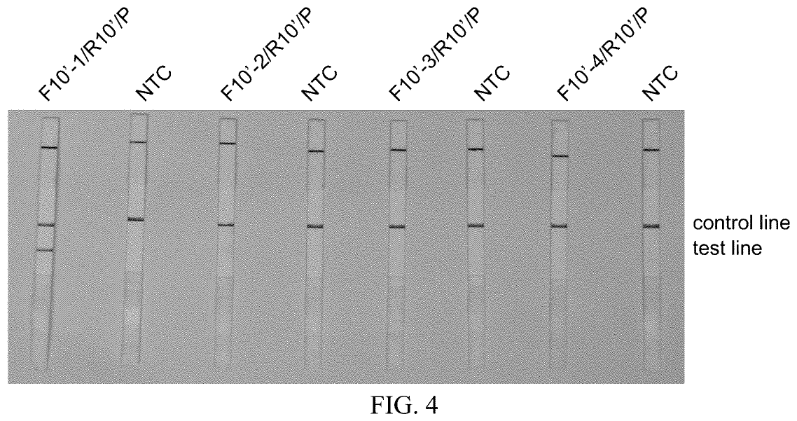

For the screening of the optimal primer-probe set, the 4 combinations described in Table 5 (F10′-1/R10′/P, F10′-2/R10′/P, F10′-3/R10′/P, and F10′-4/R10′/P) were subjected to test and screening, and the results are shown in FIG. 4. The RAA-LFS results show that only F10′-1/R10′/P had the highest amplification efficiency in the four combinations and had no false-positive NTC results, namely the F10′-1/R10′/P combination had the highest amplification efficiency and no obvious false positive, meeting the detection requirement. This result is a technical effect that those skilled in the art would have not expected before experimental verification. Thus F10′-1/R10′/P was used as the optimal primer-probe combination in subsequent experiments.

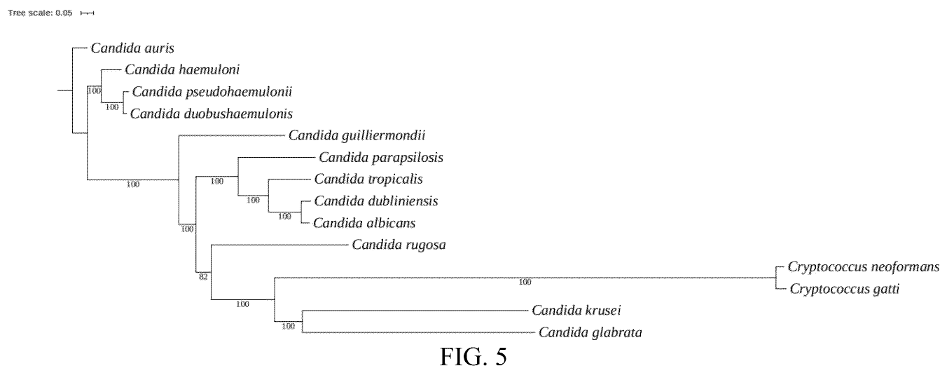

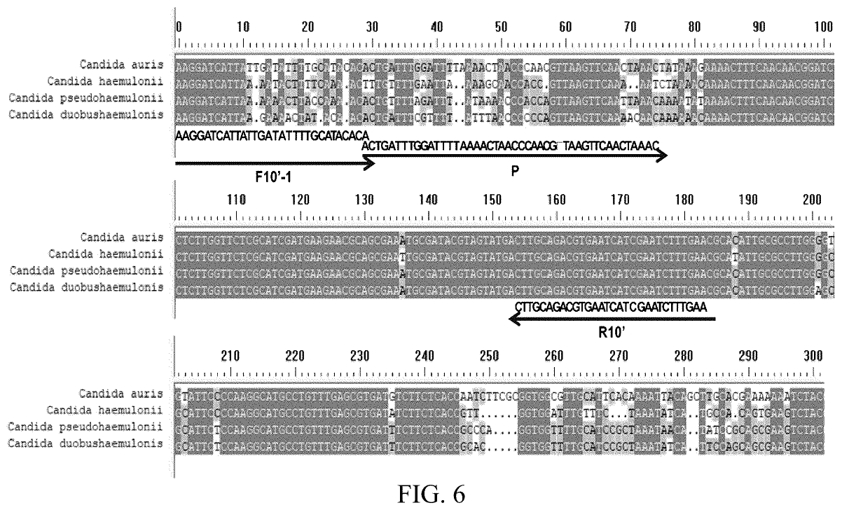

In addition, phylogenetic trees of the whole genome of Candida auris and other common pathogen Candida (shown in FIG. 5) were presented and compared with ITS sequences of three Candida with the closest relationship. The primer probe combination F10′-1/R10′/P obtained through screening can detect and distinguish Candida auris (C.auris) from C. persedohaemulonii, C. duobushaemulonis, and C. haemulonii (shown in FIG. 6), which further indicates that the primer-probe combination obtained through design and screening of the present application has a relatively high specificity.

### Example 2. Screening of Optimal Reaction Conditions for Candida Auris RAA Detection

### 1. Method

In order to screen for the optimal reaction parameters of the experiment, the genome concentration of 10³ fg/reaction was selected, and the same reaction system was placed at different reaction temperatures for testing. The specific screening method was as follows:

In the reaction, Candida auris genomic DNA with the template concentration of 10³ fg/reaction was selected. For the trial and verification of the reaction conditions, the reaction temperature was set to be 35-45° C. (one gradient was set at every 2° C.), the reaction time was set to be 5-35 min (one gradient was set at every 5 min), and incubation was separately performed. Combining the strip results, the range of the suitable reaction temperature was analyzed, and the experimental method was the same as that of 1.2.3.4 in Example 1.

### 2. Results

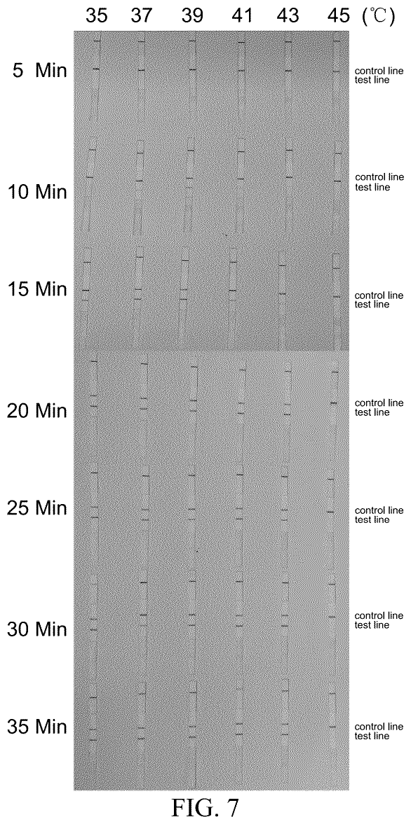

The amplification result was analyzed by LFS. The experimental result shows that pink color on the test line did not appear under the reaction condition of 45° C., possibly due to the enzyme being inactivated by the high temperature. The pink color on the test lines of the remaining strips appeared at 39° C. and 10 min, and at 15 min the color of each band began to darken, with the bands at 37° C. and 39° C. being most pronounced. After 20 minutes, the darkness of the strip did not change significantly (as shown in FIG. 7). Therefore, 37° C. and 15 min were chosen as the optimal reaction temperature and time for RAA.

### Example 3. Specificity Evaluation of RAA-LFS Technology for Detecting Candida Auris

### 1. Method

To confirm the inclusiveness and specificity of the primer-probe set, the RAA-LFS amplification tests were performed on 12 Candida auris reference strains, 4 C.haemulonii strains, 3 C.pseudohaemulonii strains, 4 C.duobushaemulonis strains, other closely related Candida, and common pathogenic bacteria in the same manner as 1.2.4.4 in Example 1, i.e., the RAA reaction was performed using the RAA-nfo nucleic acid amplification reagent (Hangzhou Zhongce Bio-Sci & Tech Co.,Ltd., Hangzhou, China) according to the manufacturer's instructions. The RAA reaction process was as follows: The reaction system was 50 μL in total, into a detection unit tube comprising an RAA reaction dry powder reagent were added 2 μL of each of 2 μM upstream and downstream primers, 0.6 μL of a 2 μM probe, 25 μL of A Buffer, 15.9 μL of double distilled water (ddH₂O), and 2 μL of DNA template, and 2.5 μL of B Buffer was added on the tube cover. The tube cover was covered. The tube was turned upside down, gently shaken, and mixed well for 5-6 times. The mixture was added into the system by centrifugation for catalyzing the reaction. After centrifugation at low speed for 10 s, the mixture was incubated in a PCR instrument (or a constant-temperature metal bath or water bath pot) at 37° C. for 15 min. After the reaction was completed, 50 μL of the amplification product was added to 300 μL of sterile water or 1×PBS solution for dilution. A disposable nucleic acid detection strip (lateral flow strip) (Hangzhou Zhongce Bio-Sci & Tech Co.,Ltd., Hangzhou, China) was inserted with the sample pad facing down into the diluted reaction solution for 2 minutes. After the sample was absorbed to the absorbent pad, the result was visually interpreted. The result interpretation method was the same as described in Example 1.

### 2. Results

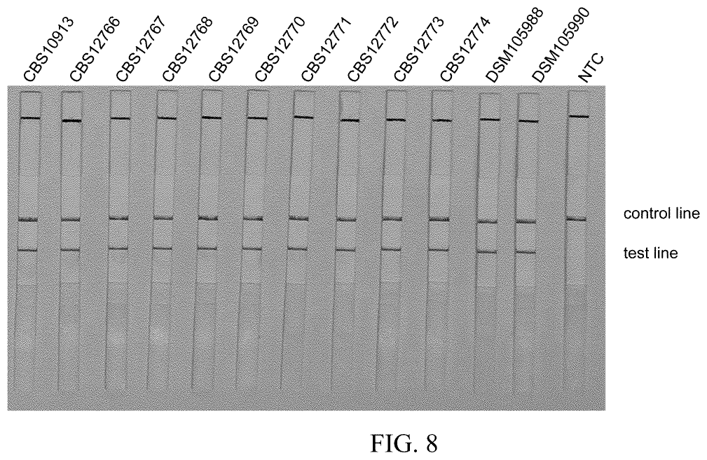

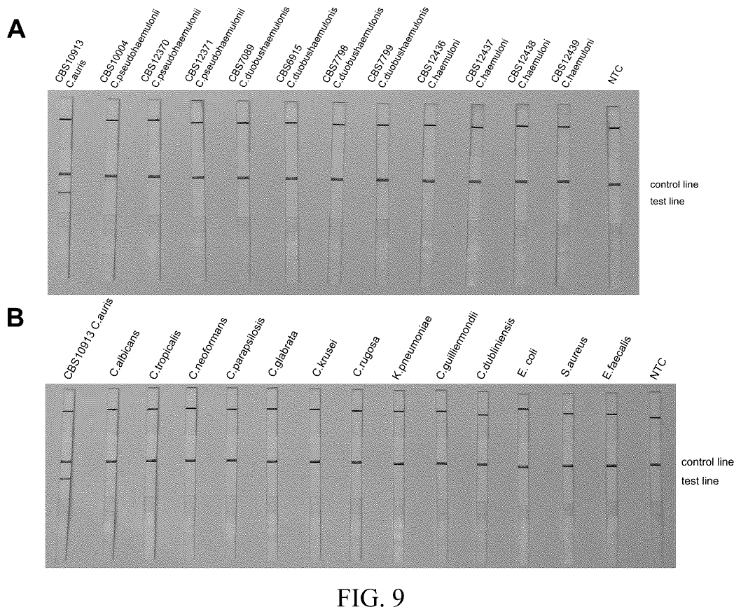

The Candida auris results for the 12 reference strains (as shown in FIG. 8) were positive, and the results for other closely related fungi and pathogenic bacteria (as shown in FIG. 9 panels A and B) were negative, indicating that the F10′-1/R10′/P primer-probe set exhibited good inclusiveness and specificity for Candida auris and did not cross-react with other pathogenic bacteria and fungi. All of C.haemulonii, C.pseudohaemulonii, and C.duobushaemulonis were negative in the test (as shown in FIG. 9 panel A), indicating that the system can detect Candida auris while accurately distinguishing them from C.haemulonii, C.pseudohaemulonii, and C.duobushaemulonis, which are excellent in specificity.

### Example 4. Sensitivity Evaluation of RAA-LFS Technology for Detecting Candida Auris

### 1. Method

In order to determine the detection limit of the RAA-LFS detection system on Candida auris, the 10-fold serial dilution of Candida auris genome and bacterial solution was tested. The genome concentration gradient was 10⁷ fg/reaction-10⁰ fg/reaction, and the bacterial solution concentration gradient was 10⁵ CFU/μL-10⁰ CFU/μL (reaction volume: 50 μL, 2 μL C.auris genome was added to each reaction). Meanwhile, in order to determine whether the contamination of other strains can interfere the detection sensitivity, the culture after treatment of 10⁵ CFU/μL Candida albicans or 1 ng/μL genomic DNA was added to a 10-fold diluted Candida auris culture (10⁵-10⁰ C. FU/μL) or genomic DNA (10⁷ fg/reaction-10⁰ fg/reaction), and the experimental method was the same as the RAA reaction process described in Example 3.

### 2. Results

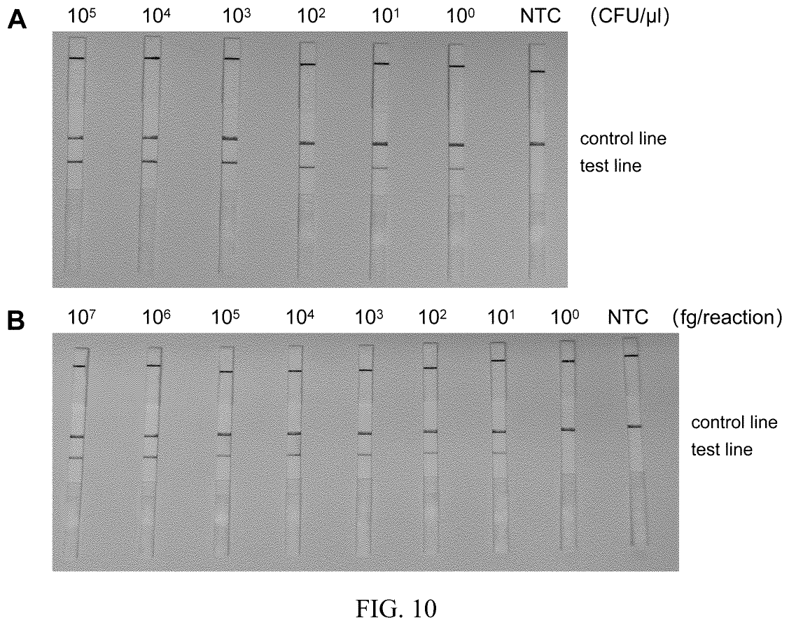

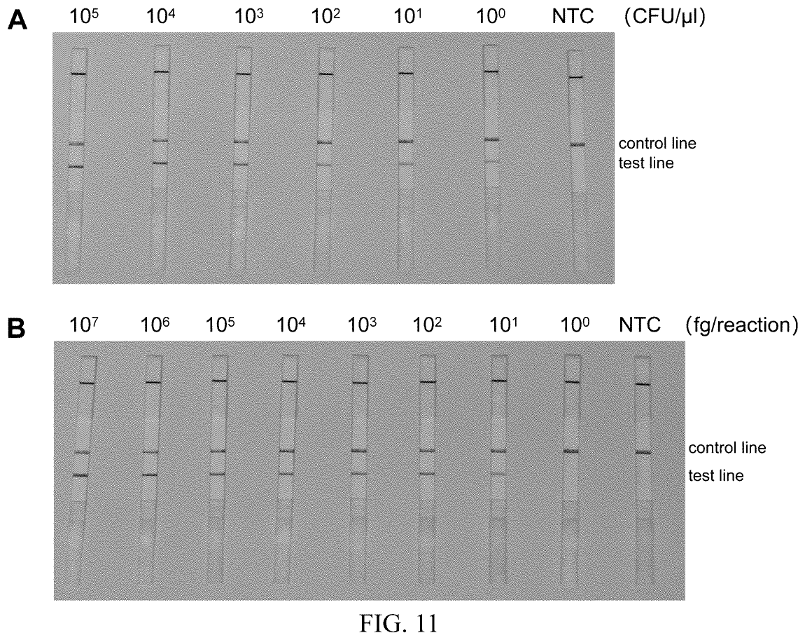

In order to determine the detection limit of the RAA-LFS system on Candida auris, in this example, the 10-fold serial dilution of Candida auris bacterial solution was tested, ranging from 10⁵ to 10⁰ CFU/μL (reaction volume: 50 μL, 2 μL Candida auris genome was added to each reaction). Despite the lighter color, a pink band still appeared on the 100 CFU/μL test line. Furthermore, as the concentration of Candida auris bacterial solution increased, the pink band became dark (as shown in FIG. 10 panel A). In a similar manner, a 10-fold serial dilution of purified Candida auris genomic DNA was performed. Candida auris genomic DNA as low as 10¹ fg/reaction can be detected (as shown in FIG. 10 panel B). In order to test whether the system can resist the interference of DNA of other common pathogenic fungi, 10⁵ CFU/μL of Candida albicans bacterial solution or 1 ng/μL genomic DNA was added to 10-fold diluted Candida auris bacterial solution (10⁵-10⁰ CFU/μL) or genomic DNA (10⁷ fg/reaction-10⁰ fg/reaction). The result shows that C.albicans bacterial solution (as shown in FIG. 11 panel A) or genomic DNA (as shown in FIG. 11 panel B) did not interfere the detection of Candida auris by RAA-LFS. It can be seen that the detection limit of the RAA-LFS system was 2 CFU per reaction, or 10 fg genomic DNA/50 μL. The detection sensitivity was not influenced by the existence of other fungal DNA.

### Example 5. Applicability Evaluation of RAA-LFS Technology for Detecting Candida Auris

### 1. Method

(1) Incorporating Candida auris Genomic DNA at Various Concentrations into Blood DNA

In order to determine the influence of human DNA on Candida auris detection, Candida auris genomic DNA and human blood-extracted DNA were mixed well according to a ratio of 1:10 and then subjected to a 10-fold serial dilution gradient, so that the final concentration of the Candida auris genome was 10⁷ fg/reaction-10⁰ fg/reaction for RAA-LFS detection. The change of the detection limit was observed, and the experimental method was the same as the RAA reaction process described in Example 3.

(2) Direct Detection of Candida auris in the Blood

The DNA was directly extracted from the whole blood incorporated with Candida auris for RAA detection, and the application of the DNA to clinical blood sample rapid detection was preliminarily evaluated. The specific steps were as follows: The RAA-LFS detection was performed by incorporating Candida auris 10-fold serial dilution bacterial solution per 200 μL whole blood sample. The bacterial solution was at a concentration gradient of 10⁵ CFU/μL to 10⁰ CFU/μL, and the experimental method was the same as the RAA reaction process described in Example 3.

### 2. Results

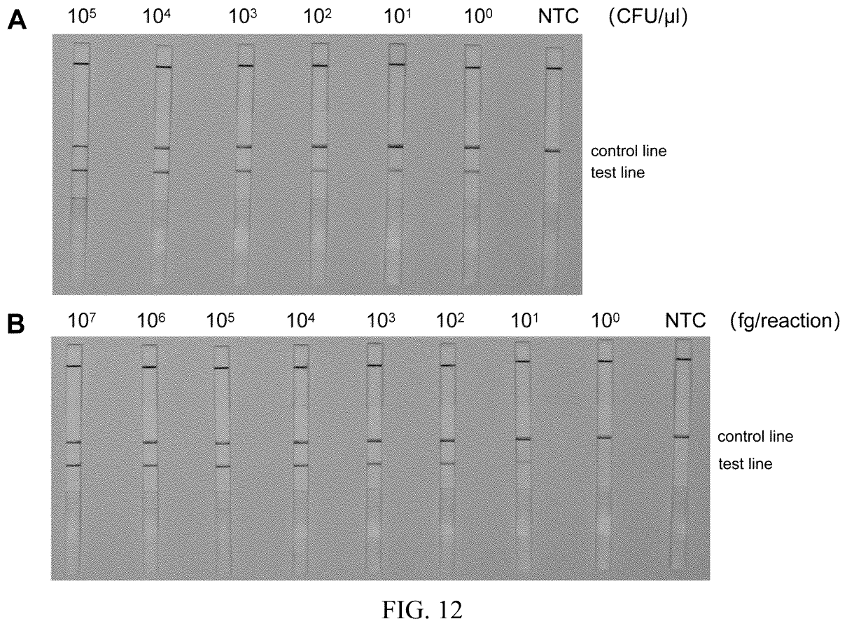

In this example, clinical blood samples were simulated. Candida auris bacterial solution at different concentrations was incorporated in human blood samples for RAA-LFS detection. The experimental results show that: The method for detecting Candida auris by RAA-LFS can be applied to a human blood sample, and the detection sensitivity was unchanged (as shown in FIG. 12 panel A); meanwhile, in order to determine the influence of human DNA on Candida auris detection, the Candida auris genomic DNA and human blood-extracted DNA were mixed well according to a ratio of 1:10 for RAA-LFS detection, and the result shows that human DNA had no significant inhibition effect on the detection and did not change the detection limit (as shown in FIG. 12 panel B).

The foregoing examples are merely intended to understand the method and core idea of the present application. It should be noted that for those of ordinary skill in the art, without departing from the principle of the present application, several improvements and modifications can also be made to the present application, and these improvements and modifications also fall within the protection scope of the claims of the present application.

1. A constant temperature nucleic acid amplification RAA primer probe combination for detecting Candida auris, wherein the primer probe combination comprises an upstream primer, a downstream primer, and a probe;
the upstream primer has a nucleotide sequence of F10′-1: 5′-AAGGATCATTATTGATATTTTGCATACACA-3′;
the downstream primer has a nucleotide sequence of R10′: 5′-Biotin-TTCAAAGATTCGATGATTCACGTCTGCAAG-3′;
the probe has a nucleotide sequence of P: 5′-FAM-ACTGATTTGGATTTTAAAACTAACCCAACG [THF] TAAGTTCAACTAAAC-C3spacer-3′.

2. A kit for detecting Candida auris, wherein the kit comprises a primer probe mixture consisting of the primer probe combination according to claim 1.

3. The kit according to claim 2, wherein the kit further comprises A Buffer, B Buffer, an RAA reaction dry powder reagent, and ddH₂O.

4. The kit according to claim 3, wherein the A Buffer is 20% PEG.

5. The kit according to claim 3, wherein the B Buffer is 280 mM MgAc.

6. The kit according to claim 3, wherein the RAA reaction dry powder reagent comprises the following components: dNTP, SSB protein, recA recombinase protein or Rad51, Bsu DNA polymerase, Tricine, PEG, dithiothreitol, creatine kinase, and Nfo endonuclease.

7. The kit according to claim 6, wherein concentrations of each of the components in the RAA reaction dry powder reagent are: 1 mmol/L dNTP, 90 ng/μL SSB protein, 120 ng/μL recA recombinase protein or 30 ng/μL Rad51, 30 ng/μL Bsu DNA polymerase, 100 mmol/L Tricine, 20% PEG, 5 mmol/L dithiothreitol, and 100 ng/μL creatine kinase.

8. The kit according to claim 3, wherein the kit further comprises a lateral flow strip.

9. A method for detecting Candida auris based on RAA-lateral flow chromatography technology, wherein the method comprises the following steps:
(1) extracting DNA of a test sample;
(2) performing an RAA amplification reaction using the DNA of the test sample as a template to give an amplification product, wherein an upstream primer of the RAA amplification has a nucleotide sequence of F10′-1: 5′-AAGGATCATTATTGATATTTTGCATACACA-3′; a downstream primer has a nucleotide sequence of R10′: 5′-Biotin-TTCAAAGATTCGATGATTCACGTCTGCAAG-3′; a probe has a nucleotide sequence of P: 5′-FAM-ACTGATTTGGATTTTAAAACTAACCCAACG [THF] TAAGTTCAACTAAAC-C3spacer-3′;
(3) detecting the amplification product using a lateral flow strip, wherein when two red bands appear on the strip, one in a quality control area and one in a detection area, the result is positive, indicating that the sample comprises Candida auris; when only one red band appears in the quality control area of the strip and the detection area has no band, the result is negative, indicating that the sample does not comprise Candida auris.

10. The method according to claim 9, wherein the amplification reaction in step (2) comprises the steps of: adding the upstream primer, the downstream primer, the probe, A Buffer, ddH₂O, and the template into a detection unit tube comprising an RAA reaction dry powder reagent, adding B Buffer on the tube cover, putting on the tube cover, mixing well, centrifuging at low speed, and continuing to perform the amplification reaction to give the amplification product.

11. The method according to claim 10, wherein the amounts of each of the substances used are: 2 μL of 2 μM upstream primer, 2 μL of 2 μM downstream primer, 0.6 μL of 2 μM probe, 25 μL of A Buffer, 15.9 μL of ddH₂O, 2 μL of template, and 2.5 μL of B Buffer, respectively.

12. The method according to claim 11, wherein the reaction conditions of the amplification reaction in step (2) are: reacting at 37° C. for 15 min.

13. The method according to claim 9, wherein the detecting Candida auris comprises detecting Candida auris and/or identifying and diagnosing Candida auris, C. persedohaemulonii, C. duobushaemulonis, and C. haemulonii.

14-16. (canceled)

17. A method for diagnosing whether a subject has a Candida auris infection disease, wherein the method comprises the step of: detecting a test sample derived from the subject using the method according to claim 9.
